(19) United States
(12) Patent Application Publication
(10) Pub. No.: US 2015/0141602 A1
(43) Pub. Date: May 21, 2015

(54) PROCESS FOR TREATING A DICYCLOPENTADIENE MONOMER

(71) Applicant: Dow Global Technologies LLC, Midland, MI (US)

(72) Inventors: Marvin L. Dettloff, Lake Jackson, TX (US); Richard J. Keaton, Pearland, TX (US); Ha Q. Pham, Lake Jackson, TX (US); Nikhil E. Verghese, Lake Jackson, TX (US)

(21) Appl. No.: 14/399,413

(22) PCT Filed: Apr. 16, 2013
(86) PCT No.: PCT/US2013/036676

§ 371 Date: Nov. 6, 2014

Related U.S. Application Data
(60) Provisional application No. 61/649,965, filed on May 22, 2012.

(51) Int. Cl. C08F 32/08; C07C 7/00; C07C 7/148

(52) U.S. Cl. CPC C08F 32/08; C07C 7/1485; C07C 7/005; C07C 2103/68
USPC 526/171; 585/836; 585/803

(57) ABSTRACT

A monomer treatment process including treating at least one metathesis polymerizable monomer composition having a purity of less than 95 weight percent of a dicyclopentadiene monomer with an alkali metal-containing additive prior to polymerizing the metathesis polymerizable monomer composition such that the treated polymerized monomer exhibits improved properties in metathesis reactions.

### FIELD

The present invention is related to a process for treating a metathesis polymerizable monomer composition such as a dicyclopentadiene monomer-containing composition such that when the treated polymerizable monomer composition is polymerized, the composition exhibits advantageous properties.

### BACKGROUND

In ring opening metathesis polymerization (ROMP) of dicyclopentadiene (DCPD), a monomer stream containing more than 98 percent (%) DCPD monomer must be used in order to: (a) use less DCPD monomer polymerization catalyst relative to a monomer stream containing less than 98% DCPD monomer, and (b) obtain a polymerized polymer such as polymerized DCPD (polyDCPD) having better mechanical properties relative to a monomer stream containing less than 98% DCPD monomer (e.g., higher glass transition temperature, and higher modulus).

U.S. Pat. No. 6,020,443 discloses a method for synthesizing polyDCPD via ROMP of low grade DCPD starting materials using ruthenium or osmium carbene complex catalyst. The low grade DCPD starting materials contain less than 97% by weight of DCPD monomers. U.S. Pat. No. 6,020,443 does not provide a treatment comprising an additive to modify any of the starting materials prior to polymerization.

It would be desirable to have a ROMP process that does not require high levels (for example greater than (>) 1500 ppm) of polymerization catalyst. It would also be desirable to have a treatment process for treating a particular grade of monomer stream, such as DCPD monomer stream, containing less than 98% DCPD monomer, that would provide a polymerized product with improved performance properties such as a higher Tg, and less brittleness, compared to a polymerized product made from the untreated version of the same grade of DCPD monomer.

### SUMMARY

To overcome the problems of the prior art, a monomer treatment process has been developed which improves monomer conversion and achieves polymerized products having higher Tg values compared to control runs performed without the treatment process of the present invention. In addition, one of the improvements in the present invention is the use of less catalyst in a polymerization process using a treated monomer material versus the untreated form of the monomer material.

One embodiment of the present invention is directed to a monomer treatment process including the step of treating a dicyclopentadiene monomer with an alkali metal-containing additive prior to polymerizing the monomer.

Another embodiment of the present invention is directed to a process for polymerizing a monomer including the steps of: (a) treating a dicyclopentadiene monomer with an alkali metal-containing additive; and (b) polymerizing the treated monomer of step (a). The alkali metal-containing additive may remain in the polymerization process step (b), or can be separated from the dicyclopentadiene monomer prior to step (b).

Some of the advantages of using the treatment method of the present invention may include for example, but not limited thereto: (1) a reduction in color present in the treated monomer compared to the untreated monomer, (2) a higher Tg value exhibited by a polymerized article prepared from a treated monomer when compared to a polymerized article prepared from an untreated monomer at an equivalent amount of metathesis polymerization catalyst loading, or (3) a comparable or higher Tg value exhibited by a polymerized article prepared from a treated monomer when compared to a polymerized article prepared from an untreated monomer using a lower metathesis polymerization catalyst loading than the amount of catalyst used to polymerize untreated monomer.

### DETAILED DESCRIPTION

One broad aspect of the present invention includes a process of treating a monomer with a treatment additive, wherein the treatment additive includes, for example, an alkali metal, an oxidized alkali metal, or mixtures thereof. In one preferred embodiment, the treatment additive may be an alkali metal, an oxidized alkali metal, or mixtures thereof coated on a support. The treatment process is carried out, prior to polymerization of the monomer, under process conditions such as at a predetermined temperature and for a predetermined period of time to form a treated monomer, wherein the treated monomer can be subsequently used to form a cured resin product.

In one embodiment, the first step of the process of the present invention includes treating a monomer, such as for example a DCPD monomer, with the treatment additive at a predetermined temperature for a predetermined period of time. For example, generally, the temperature of the treating step is from 10° C. to 120° C. in one embodiment, from 15° C. to 80° C. in another embodiment, and from 20° C. to 50° C. in still another embodiment; and generally, the time of the treating step is from 1 minute to 16 hours in one embodiment, from 5 minutes to 8 hours in another embodiment, and from 20 minutes to 2 hours in still another embodiment.

In another embodiment, the treated monomer, such as for example a treated DCPD monomer which has been treated with the treatment additive, can include low grade DCPD starting materials. The process of the present invention may include for example using the treated monomer in a method for synthesizing a polymer such as polyDCPD via ROMP of the low grade DCPD starting material in combination with a ROMP catalyst such as a ruthenium or osmium carbene complex catalyst, or other catalysts based on tungsten, molybdenum and titanium such as described in U.S. Pat. Nos. 4,661,575; 4,952,348; 4,994,426; and 5,319,042, each of which is incorporated herein by reference.

The DCPD monomer useful for the polymerization process in accordance with the present invention is typically produced as a result of a process involving the high temperature cracking of petroleum fractions to make ethylene such as described by Cheung, T.T.P. 2001 “Cyclopentadiene and Dicyclopentadiene”, Kirk-Othmer Encyclopedia of Chemical Technology. Depending on the particular producer, different grades of DCPD monomer are sold commercially. For example, three different grades of DCPD monomer are commercially available from The Dow Chemical Company as described in Product Data Sheet for Dicyclopentadiene (DCPD) (Dow Form #778-00101, published August 2005) available from The Dow Chemical Company as well as brochure entitled “Dicyclopentadiene Products A Guide to Product Handling and Use (Dow Form #778-04301, created May 2010) also available from The Dow Chemical Company. The three grades of DCPD commercially available from The Dow Chemical Company are listed in the following table.

In general, the purity of the dicylopentadiene monomer useful in the present invention is less than 95 wt %, preferably from 10 wt % to 95 wt %, more preferably from 20 wt % to 95 wt %, even more preferable from 40 wt % to 95 wt %, and most preferably from 50 wt % to 95 wt %.

Typical DCPD Product Grades

| | DCPD RESIN | DCPD, UPR | DCPD HIGH |
|---|---|---|---|
| Component | GRADE | GRADE | PURITY |
| Endo-DCPD | 73-83% | 83-88% | 90-95% |
| Exo-DCPD | 0.5-1% | 0.5-1% | 0-1% |
| CPD-mCPD codimer | 7-13% | 2-6% | 0-1% |
| Isoprene-CPD codimer | 1-5% | 1-5% | 1-5-% |
| Butadiene-CPD codimer | 0-5% | 0-5% | 0-1% |
| Piperylene-CPD codimer | 0-2% | 0-2% | 0-1% |
| CPD-trimer | 0-1% | 0-0.1% | 0-1% |
| CPD | 0-1% | 0-1% | 0-1% |
| m-Bicyclononadiene | 0-2% | 0-2% | 0-1% |
| Benzene | <0.01% | <0.01% | <0.01% |
| Toluene | <0.02% | <0.01% | <0.0005% |

DCPD = dicyclopentadiene
CPD = cyclopentadiene
UPR = unsaturated polyester resin

Higher purity grades are also commercially available. For example, Ultrene 97 (≧97% DCPD monomer) and Ultrene 99 (≧99% DCPD monomer) are commercially available from Cymetech.

Generally, the DCPD monomer useful in the present invention can include a crude DCPD monomer having a purity of less than (<)95% purity in one embodiment, less than 92% in another embodiment, and less than 88% in still another embodiment. In another embodiment, the purity of the DCPD monomer used in the present invention may be from 75% to 100%, from 80% to 95% in still another embodiment, and from 83% to 92% in yet another embodiment.

The type of catalysts employed in the present invention to treat the monomers may include for example the heterogeneous form of a base as described in http://signachem.com/products/by-name/alkali-metal-alumina-gel/; or in Oh et al., Bull. Korean Chem. Soc., 2008, 29(11), 2202-2203, which discloses the isomerization of 5-vinyl-2-norbomene using sodium-coated catalysts. In the present invention some olefin isomerization occurs and simultaneously, trace impurities such as the sulfur compounds are removed with the use of a supported strong base. For example, the types of strong bases useful in the present invention are described in EP 279397; U.S. Pat. No. 5,981,820; WO 94/24076; and WO 00/18710.

The treatment additive useful for treating the DCPD monomer of the present invention can be an alkali metal, an alkali metal oxide, or a mixture thereof coated on a support substrate in one embodiment. In one preferred embodiment, for example, the treatment additive may include a sodium or potassium metal and/or metal oxide coated on a support such as alumina. In another embodiment, the treatment additive may include, for example, an oxidized alkali metal such as for example Na₂O, K₂O, or a mixture thereof. The treatment additive may be used alone or may be used in a combination of two or more treatment additives.

In another embodiment, the treatment additive may be coated on a solid support member such as alumina, silica, carbon, zeolites, magnesium chloride, magnesium oxide, clays, nano-clays, or mixtures thereof. In a preferred embodiment, the solid support includes an alumina, a silica, or a mixture thereof.

Generally, the amount of the treatment additive used in the present invention may be in the range of from 0.1 weight percent (wt %) to 20 wt % in one embodiment, from 0.3 wt % to 8 wt % in another embodiment, and from 0.5 wt % to 6 wt % in still another embodiment, based on the total amount of metathesis polymerizable monomer.

The treatment process of the present invention for treating a DCPD monomer may optionally include for example adding a catalyst activity modifying agent to the monomer to moderate catalyst activity. For example the catalyst activity modifying agent may include a phosphine, a silane, a pyridine, a tertiary amine, or mixtures thereof.

Generally, the amount of catalyst activity modifying agent, when used, may be in the range of from 0 wt % to 1.0 wt % in one embodiment, from 0.02 wt % to 0.6 wt % in another embodiment, and from 0.04 wt % to 0.2 wt % in still another embodiment.

After the first step of treating a monomer with treatment additive, the treated monomer may optionally be followed by a step of separating the treated monomer from the treatment additive compound. Separating the treated monomer may be carried out by a variety of recovery techniques such as for example filtration, centrifugation, and distillation.

In one preferred embodiment, the treated monomer may be recovered by using known filtration processes and equipment. For example, the process of the present invention may include filtering the treated monomer by various filtration processes and equipment to isolate the treated monomer from the other compounds remaining after the treatment step.

In another embodiment, the treatment additive such as an alkali metal additive and other additives may remain with the treated monomer after the treatment step without separating (for example, by filtration) the treated monomer from the treatment additive and other additives. In one embodiment, the resulting unfiltered treated monomer material can be directly polymerized, for example in a metathesis reaction, to form the polymerized product without a detrimental effect on the properties of the resultant polymerized product.

Another optional step that may be included in the process of the present invention is a degassing step. Degassing a monomer useful in the present invention process may be done at any point or step of the process such as for example, before or after the treatment process step; before or after the separating step (e.g. filtering); or before the polymerization step.

Degassing the monomer involves removal of dissolved gases in the monomer such as air and may be carried out by a variety of degassing techniques such as for example inert gas sparging, low pressure evacuation, freeze/pump/thaw cycles, or combinations thereof.

The treated monomer of the present invention may exhibit several improved properties including reduced levels of nitrogen-containing compounds; reduced levels of sulfur-containing compounds; and/or reduced levels of 5-vinyl-2-norbornene (VNB), among others. It is important to reduce the levels of the above compounds because the compounds may (a) deactivate or inhibit the activation of the metathesis catalyst/initiator, (b) deactivate or inhibit the activity of the metathesis catalyst/initiator, (c) alter the molecular weight (MW) growth and cross-link density of a cured product produced from a curable composition containing the compounds, and/or (d) lead to a cured product with significant brittle characteristics.

For example, generally the level of nitrogen-containing compounds in the treated monomer of the present invention may be from less than 200 ppm in one embodiment, and from less than 10 ppm in another embodiment.

For example, generally the level of sulfur-containing compounds in the treated monomer of the present invention may be less than 100 ppm in one embodiment, from 50 ppm to 1 ppm in another embodiment, and from 10 ppm to less than 1 ppm in still another embodiment.

For example, generally the level of VNB in the treated monomer of the present invention may be less than 1.5 wt % in one embodiment, from 1 wt % to 0.2 wt % in another embodiment, and from 0.9 wt % to less than 0.1 wt % in still another embodiment.

Another broad aspect of the present invention includes a process for polymerizing the treated monomer such as a treated DCPD product. For example, the treated DCPD product may be subjected to a ROMP reaction. Generally, the DCPD monomer stream is a treated DCPD having a purity of less than 95% DCPD purity in the ROMP reaction process. One advantage of the present invention is the flexibility to perform a ROMP reaction process using DCPD regardless of the purity level of the DCPD grade.

The polymerization reaction mixture includes treated DCPD, initiators (or catalysts), co-catalysts, additional monomers capable of undergoing a metathesis reaction, reactivity control agents (for example as described in U.S. Pat. No. 5,939,504 or U.S. Pat. No. 7,060,769), viscosity modifiers, surfactants, fillers, dyes, solvents or mixtures thereof.

For example, as an illustrative embodiment, when a Ru catalyst is employed in the polymerization process, the concentration of the catalyst may be reduced from 2500 ppm in the untreated DCPD monomer to 1500 ppm using the same grade of treated DCPD monomer in one embodiment, from 1500 ppm in the untreated DCPD monomer to 750 ppm using the same grade of treated DCPD monomer in another embodiment, from 750 ppm in the untreated DCPD monomer to 300 ppm using the same grade of treated DCPD monomer in still another embodiment, and from 300 ppm to 40 ppm using the same grade of treated DCPD monomer in yet another embodiment.

The process of polymerizing a DCPD monomer useful in the present invention, such as for example a ROMP process, is known in the art. The treated DCPD monomer can undergo ROMP polymerization by any of the methods known in the art including for example processes involving a ruthenium or an osmium-based catalyst such as described in CA 2246789; U.S. Pat. Nos. 5,728,785; 5,939,504; 6,020,443; 6,310,121; 6,323,296; 6,410,110; 6,750,272; 7,285,593; 7,339,006; and 7,700,698; U.S. Patent Application Publication Nos. 20090061713; 20090062441; 20090062446; 20090156726; and 20090156735; JP 2009143156; JP 2001026059; and WO 2011005136.

### EXAMPLES

The following examples and comparative examples further illustrate the present invention in detail but are not to be construed to limit the scope thereof.

Various terms and designations used in the following examples are explained herein below:

“DCPD” stands for dicyclopentadiene.

“VNB” stands for 5-vinyl-2-norbomene.

The monomers used in the Examples include DCPD UPRG (83-88 wt % DCPD), DCPD HP (90-95 wt % DCPD), and Ultrene DCPD (>98 wt % DCPD).

“UPR” stands for unsaturated polyester resin.

DCPD UPRG is UPR Grade and commercially available from The Dow Chemical Company.

“HP” stands for high purity.

DCPD HP is DCPD High Purity Grade and commercially available from The Dow Chemical Company.

Ultrene DCPD is a DCPD product commercially available from Cymetech.

The structures of two ruthenium (Ru) initiators employed in the Examples are shown in the following Structures (I) and (II), referred to as CAT1 and CAT2, respectively:

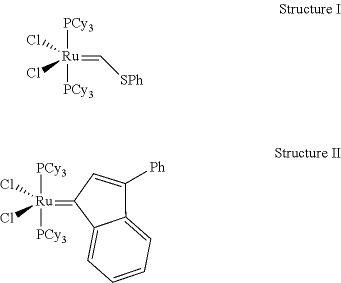

CAT1 (Structure I) is bis(tricyclohexylphosphine)[(phenylthio) methylene]ruthenium(II) dichloride and commercially available from Strem Chemicals, Inc. CAT2 (Structure II) is bis(tricyclohexylphosphine)-3-phenyl-1H-inden-1-ylideneruthenium(II) dichloride and commercially available from Strem Chemicals, Inc.

“DSC” stands for differential scanning calorimetry or calorimeter.

“TGA” stands for thermogravimetric analysis.

The following standard analytical equipment and methods are used in the Examples:

General Procedure for Preparing Catalyst Mixtures

A targeted wt % catalyst solution was prepared by adding an appropriate amount of a solid catalyst to methylcyclohexane (MCH) (which had previously been passed through a column of activated molecular sieves). The catalyst mixture was added to a DCPD monomer after inserting a microliter pipetman tip into an actively stirring catalyst mixture to homogeneously disperse the catalyst in the MCH.

General Treatment Procedure

An appropriate DCPD feedstock was mixed at about 23° C. overnight with a treatment additive. This mixture was then filtered to remove any residual solids.

General DCPD Curing Procedure

Inside a nitrogen-filled glove box, 2.0 g of an appropriate DCPD grade was added to a 4 mL vial containing a stir bar. An appropriate amount of catalyst (as a mixture in MCH) was added to the vial with stifling. The vial was sealed, removed from the glove box, and heated at a target temperature for a given time (typically, 70° C. for two hours). Once the curing was complete, the vial was transferred into an oven and post-cured at a target temperature for a given time (typically, 120° C. for two hours). A sample for DSC or TGA analysis was trimmed from the top of a cured plug after breaking the vial and removing glass shards.

DSC Measurements

A sample of approximately 6 mg to 9 mg was cut from a cured piece and loaded into an aluminum pan that was then hermetically sealed. The pan was loaded into an autosampler on a TA Instruments D200 DSC. The sample was cooled to 25° C., ramped at 10° C./min to 225° C., equilibrated again at 25° C., then ramped a second time to 225° C. at a rate of 10° C./min.

TGA Measurements

A sample of approximately 7 mg to 10 mg were cut from a cured piece and loaded into a tared 100 ml platinum pan containing a disposable DSC aluminum pan. The pan was loaded into an autosampler on a TA Instruments Q5000 TGA. The sample was ramped at 10° C./min from ambient conditions to 350° C.

### Examples 1 and 2

In each of Examples 1 and 2, 2.0 g of DCPD HP grade was mixed overnight with 1.0 wt % Na silica gel Stage 1 (from SIGNa Chemistry, Inc. through Sigma-Aldrich), and then mixed overnight with 1.0 wt % NaO/Na on alumina (from SIGNa Chemistry, Inc. through Sigma-Aldrich), followed by filtration to form a treated sample of DCPD HP grade.

Inside a nitrogen-filled glovebox, each of the 2.0 g of treated DCPD HP grade was added to a 4 mL vial containing a stir bar. A CAT1 catalyst mixture was added (as a 10 wt % dispersion in methylcyclohexane) to the vial with stirring. The vial was sealed, removed from the glove box, and heated at 70° C. for two hours. The vial was then post-cured at 120° C. for two hours. The results of Examples 1 and 2 are described in Table I.

### Examples 3 and 4

Each of Examples 3 and 4 was carried out using the same procedure as described in Examples 1 and 2 above except that each of the 2.0 g of DCPD HP grade was first mixed overnight with 3.3 wt % Na silica gel Stage 1 (from SIGNa through Sigma-Aldrich), and then mixed overnight with 3.3 wt % NaO/Na on alumina (from SIGNa through Sigma-Aldrich), followed by filtration. The results of Examples 3 and 4 are described in Table I.

### Comparative Example A

### Control

This Comparative Example A was carried out using the same procedure as described in Examples land 2 above except that the 2.0 g of DCPD HP grade was not first treated overnight with 1.0 wt % Na silica gel Stage 1 or 1.0 wt % NaO/Na on alumina. Instead, the untreated 2.0 g of DCPD HP grade was added to a 4 mL vial containing a stir bar and prepared as described in Examples land 2 above. The results of this Comparative Example A are described in Table I.

TABLE I
DCPD Feedstock Monomers Cured

| | | | | | | Post | |
|---|---|---|---|---|---|---|---|
| | | Treatment | | Catalyst | Cure | Cure | DSC |
| Exam- | DCPD | Additive | Catalyst | Amount | Temp | Temp | Tg |
| ple | Used | Used | Used | (ppm) | (° C.) | (° C.) | (° C.) |
| Exam- | HP | 1.0% Na | 10 wt % | 1150 | 70 | 120 | 131.4 |
| ple 1 | | silica gel | Cat1 in | | | | |
| | | Stage 1 then | MCH | | | | |
| | | 1.0% | | | | | |
| | | NaO/Na (on | | | | | |
| | | alumina) | | | | | |
| Exam- | HP | 1.0% Na | 10 wt % | 1550 | 70 | 120 | 132.5 |
| ple 2 | | silica gel | Cat1 in | | | | |
| | | Stage 1 then | MCH | | | | |
| | | 1.0% | | | | | |
| | | NaO/Na (on | | | | | |
| | | alumina) | | | | | |
| Exam- | HP | 3.3% Na | 10 wt % | 770 | 70 | 120 | 137.9 |
| ple 3 | | silica gel | Cat1 in | | | | |
| | | Stage 1 then | MCH | | | | |
| | | 3.3% | | | | | |
| | | NaO/Na (on | | | | | |
| | | alumina) | | | | | |
| Exam- | HP | 3.3% Na | 10 wt % | 1150 | 70 | 120 | 137.9 |
| ple 4 | | silica gel | Cat1 in | | | | |
| | | Stage 1 then | MCH | | | | |
| | | 3.3% | | | | | |
| | | NaO/Na (on | | | | | |
| | | alumina) | | | | | |
| Com- | HP | None | 10 wt % | 1550 | 70 | 120 | 85.3 |
| para- | | | Cat1 in | | | | |
| tive | | | MCH | | | | |
| Exam- | | | | | | | |
| ple A | | | | | | | |

### Example 5

In this Example 5, 2.0 g of Ultrene DCPD grade solution was stirred overnight with 3 wt % NaO/Na on alumina (from SIGNa through Sigma-Aldrich), and then the solids were removed via filtration before addition of a catalyst.

Inside a nitrogen-filled glovebox, the 2.0 g of treated Ultrene DCPD grade was added to a 4 mL vial containing a stir bar. A CAT1 mixture (10 uL) was added (as a 10 wt % dispersion in methylcyclohexane) to the vial with stirring. The vial was sealed, removed from the glovebox, and heated at 50° C. for two hours. The vial was then post-cured at 120° C. for two hours. The results of this Example 5 are described in Table II.

### Comparative Example B

This Comparative Example B was carried out using the same procedure as described in Example 5 except that the 2.0 g of Ultrene DCPD grade was not first treated overnight with 3 wt % NaO/Na on alumina or the solids removed via filtration.

Instead, the untreated 2.0 g of Ultrene DCPD grade was added to a 4 mL vial containing a stir bar and prepared as described in Example 5 above. The results of this Comparative Example B are described in Table II.

### Comparative Example C

This Comparative Example C was carried out using the same procedure as described in Comparative Example B except that 1.8 g of Ultrene was combined with 0.2 g of VNB before addition of the catalyst. The results of this Comparative Example C are described in Table II.

TABLE II

| | | | | | Catalyst | | Post | DSC | TGA |
|---|---|---|---|---|---|---|---|---|---|
| | | Additive | Treatment | | Amount | Cure Temp | Cure | Tg | (wt loss at |
| Example | DCPD Used | Used | Additive Used | Catalyst Used | (ppm) | (° C.) | (° C.) | (° C.) | 250° C.) |
| Example 5 | Ultrene | 10% VNB | NaO/Na on | 10 wt % | 390 | 50 | 120 | 147.5 | 0.7 |
| | | | alumina | CAT1 in | | | | | |
| | | | | MCH | | | | | |
| Comparative | Ultrene | 10% HP | None | 10 wt % | 390 | 50 | 120 | 146.7 | 0.6 |
| Example B | | | | CAT1 in | | | | | |
| | | | | MCH | | | | | |
| Comparative | Ultrene | 10% VNB | None | 10 wt % | 390 | 50 | 120 | 122.3 | 1 |
| Example C | | | | CAT1 in | | | | | |
| | | | | MCH | | | | | |

As shown in the above Table II, the treatment of the DCPD using the process of the present invention (Example 5) allows the Tg of the system to be retained, similar to a control (Comparative Example B), despite the presence of an additive (VNB) which reduces the Tg (Comparative Example C). Also, the sample with VNB (Comparative Example C) is more brittle and prone to fracture, while the treatment of the present invention removes this behavior.

### Example 6 and Comparative Examples D and E

Plaques containing untreated monomer (comparative examples) and treated monomer of the present invention (Example 6) were cast in a closed mold to produce plaques for mechanical testing. The formulation components are listed in Table III. The catalyst employed was CAT2, and the loadings are given in Table III. The molds were filled with the polymerizable formulation at 23° C. and then the molds were placed in a 50° C. oven for an hour. The oven temperature was then increased to 120° C. and the mold was left for 12 hours. The plaques were demolded after cooling the mold to room temperature.

TABLE III

| | | | Catalyst | | | Ultimate | Strain |
|---|---|---|---|---|---|---|---|
| | | | Loading | Tg | Modulus | Strength | at Ult |
| Example | Monomer | Treatment | (ppm) | (° C.) | (MPa) | (MPa) | (MPa) |
| Example 6 | HP | 2% Na silica gel | 730 | 137.3 | 1987 | 56.0 | 5.4 |
| | | Stage 1 then 2% | | | | | |
| | | NaO/Na on | | | | | |
| | | alumina), filtered | | | | | |
| Comparative | Ultrene | None | 300 | 133.5 | 2004 | 58.4 | 5.6 |
| Example D | 99% | | | | | | |
| Comparative | HP | None | 1800 | 118.5 | 1979 | 54.3 | 5.3 |
| Example E | | | | | | | |

The data in Table III above shows that the treatment method of the present invention (Example 6) leads to a polymerized product with equal or superior properties to comparable plaques made with untreated monomer (Comparative Example E), and at a significantly lower catalyst loading. The improvement of the present invention allows lower purity grades of DCPD to give comparable properties to plaques produced with the commercially available highest purity (99%) DCPD (Comparative Example D).

1. A treatment process comprising treating at least one metathesis polymerizable monomer composition having a purity of less than 95 weight percent of a dicyclopentadiene monomer with a treatment additive prior to polymerizing the metathesis polymerizable monomer composition such that the treated composition being cured in a metathesis reaction uses a reduced amount of metathesis catalyst versus an untreated composition being cured.

2. The process of claim 1, wherein the treatment additive comprises an alkali metal, an oxidized alkali metal, or mixtures thereof.

3. The process of claim 2, wherein the alkali metal comprises sodium, potassium, or mixtures thereof.

4. The process of claim 2, wherein the oxidized alkali metal comprises Na₂O, K₂O, or mixtures thereof.

5. The process of claim 1, wherein the treatment additive is coated on a solid support; and the solid support is selected from the group consisting of alumina, silica, carbon, zeolites, magnesium chloride, magnesium oxide, clays, nano-clays and mixtures thereof.

6. The process of claim 1, wherein the concentration of the treatment additive ranges from 0.1 weight percent to 10 weight percent based on the total weight of the metathesis polymerizable monomer.

7. The process of claim 1, including the step of heating the monomer at a temperature of from 20° C. to 100° C. during the treating step of the monomer.

8. The process of claim 1, including degassing the monomer prior to polymerization of the monomer.

9. The process of claim 1, including separating the monomer and the treatment additive prior to polymerization of the monomer.

10. The process of claim 1, including adding an additive to the monomer to moderate catalyst activity; wherein the additive comprises a phosphine, a silane, a pyridine, a tertiary amine or mixtures thereof.

11. The process of claim 10, wherein the phosphine comprises tricyclohexylphosphine, tricyclopentylphosphine, triisopropylphosine, tripropylphosphine, tributylphosphine, triisobutylphosphine, trihexylphosphine, trioctylphosphine, tris(2-cyano-ethyl)phosphine, tris(3-hydroxypropyl)phosphine, triphenylphosphine, or mixtures thereof.

12. The process of claim 10, wherein the silane comprises tetraallyl silane, tetravinylsilane, allyltrimethylsilane, ethynyltrimethylsilane, vinyltrimethylsilane, or mixtures thereof.

13. The process of claim 10, wherein the pyridine comprises pyridine, 4-vinylpyridine, 2-vinylpyridine, 4-dimethylaminopyridine, 2-methylpyridine, 3-methylpyridine, 4-methylpyridine, 2,6-dimethylpyridine, 2,4-dimethylpyridine, 2,4,6-trimethylpyridine, 5-ethyl-2-methylpyridine, or mixtures thereof.

14. The process of claim 10, wherein the tertiary amine comprises tricyclohexylamine, tricyclopentylamine, triisopropylamine, tripropylamine, tributylamine, triisobutylamine, trihexylamine, trioctylamine, imidazoles, substituted imidazoles, or mixtures thereof.

15. A process for polymerizing a monomer comprising the steps of:
(I) treating at least one metathesis polymerizable monomer composition having a purity of less than 95 weight percent of a dicyclopentadiene monomer with a treatment additive prior to polymerizing the metathesis polymerizable monomer composition such that the treated composition being polymerized in a metathesis polymerizing reaction uses a reduced amount of metathesis catalyst versus an untreated composition being polymerized; and
(II) polymerizing the treated monomer of step (I) to form a cured product.
